(19) United States
(12) Patent Application Publication
(10) Pub. No.: US 2013/0203688 A1
(43) Pub. Date: Aug. 8, 2013

(54) SUGAR PLANT DERIVED BY-PRODUCTS AND METHODS OF PRODUCTION THEREOF

(71) Applicants: Julie Barbeau, Boucherville (CA); Geneviève Béland, Saint-Hyacinthe (CA)

(72) Inventors: Julie Barbeau, Boucherville (CA); Geneviève Béland, Saint-Hyacinthe (CA)

(73) Assignee: FÉDÉRATION DES PRODUCTEURS ACÉRI-COLES DU QUÉBEC, Longueuil, QC (CA)

(21) Appl. No.: 13/817,718

(22) PCT Filed: Aug. 19, 2011
(86) PCT No.: PCT/CA2011/000951

§ 371 Date: Feb. 19, 2013

Related U.S. Application Data
(60) Provisional application No. 61/375,444, filed on Aug. 20, 2010.

(51) Int. Cl. C07C 43/23; C07H 1/08; C07D 307/86; C07D 311/20; C07D 493/08; C07D 493/04; A61K 31/192; A61K 31/7048; A61K 31/343; A61K 31/366; A61K 31/34; A61K 31/7034; C07C 49/825; C07C 45/78; C07D 307/33; C07C 41/34; C07C 51/42

(52) U.S. Cl. CPC C07C 43/23; C07C 51/42; C07H 1/08; C07D 307/86; C07D 311/20; C07D 493/08; C07D 493/04; A61K 31/192; A61K 31/7048; A61K 31/343; A61K 31/366; A61K 31/34; A61K 31/7034; C07C 49/825; C07C 45/78; C07D 307/33; C07C 41/34
USPC 514/25; 562/476; 536/18.2; 549/462; 549/289; 549/463; 549/464; 536/18.5; 514/568; 514/469; 514/456; 514/470; 568/324; 514/689; 549/313; 514/473; 568/644; 514/721

(57) ABSTRACT

The present document describes a nutraceutical or cosmeceutical composition for the prophylaxis of an ailment comprising a therapeutically effective amount of a sugar plant syrup filtration residue in association with a pharmaceutically acceptable carrier. The present document also describes a nutraceutical or cosmeceutical composition for improving health condition of skin. The present document describes a method of producing a sugar plant syrup-derived product; wherein the improvement is characterized in the step of: collecting a sugar plant syrup residue during the production of sugar or syrup to produce a syrup-derived by-product.

### CROSS-REFERENCE TO RELATED APPLICATIONS

This application claims priority from US provisional patent application 61/375,444, filed Aug. 20; 2010, the specification of which is hereby incorporated by reference.

### BACKGROUND

(a) Field

The subject matter disclosed generally relates to nutraceutical composition for the prophylaxis of an ailment based sugar plant syrup filtration residue and a method of producing a sugar plant syrup-derived product.

(b) Related Prior Art

Products derived from the sap of the sugar maple tree Acer Saccharum are well known in the art. For many years, it has been known that the sap of the hard or sugar maple trees, known as winter sap, could be concentrated very substantially from its original, very watery and only slightly sweet condition to well known maple syrup which, when further boiled, finally reaches crystalline stage wherein the product is maple sugar. For example, U.S. Pat. No. 3,397,062 to Nessly describes carbonated beverages derived from concentrated maple sap to which flavouring may be added. Also, U.S. Pat. No. 5,424,089 to Munch et al. describes carbonated beverages prepared from non-concentrated maple sap to which flavouring ingredients may have been added.

Other products concern principally maple sugar products such as maple syrup. For example U.S. Pat. No. 6,485,763 to Jampen describes a method for producing a shelf stable, spreadable high viscosity maple syrup by adding a sucrose-cleaving enzyme.

Using maple based products appears to present several advantages for a healthy diet, as maple-sugar products contain molecules such as polyphenols as well as other natural nutrients such as vitamins and oligoelements, amino acids, peptides, organic acids that can contribute to good health. However, very little is known concerning the potential health benefits of other types of maple-based products such as products derived from the sap, the samara (fruits/seeds), leaves, roots, sapwood, heartwood, stems (twigs) and bark of any variety of Maple tree. Also, maple tree derived sugar remains a relatively little nutrient from which only recently has been found potential properties as an anti-oxidant, anticancer, antibacterial and anti-diabetic agent.

There exist numerous sugar plants used in the production of sugar and syrup. Such sugar plants include maple tree, birch tree, sugar cane, sugar beet, corn, rice, palm tree, and agave.

Therefore, there is a need for sugar plant derived products to improve or enhance health and well-being.

Therefore, there is a need for sugar plant derived sugar products to improve or enhance health and well-being.

### SUMMARY

It is an object of the present disclosure to provide novel sugar plant derived by-products and method of production thereof.

According to an embodiment, there is provided, in a method of producing a nutraceutical or cosmeceutical composition comprising a sugar plant by-product residue comprising the step of:
a) collecting a sugar plant by-product residue from the clearing during the production of sugar or syrup to produce a syrup-derived by-product residue.

The collection of the sugar plant by-product residue may be by filtration, distillation, pressing, sedimentation, sieving, precipitation or centrifugation of sugar plant syrup.

The filtration may be effected using a filter containing one of diatomaceous earth, celite, kieselguhr, silica, silicon dioxide, calcium, natural sugar sand, ground bones, slop, clay and the like.

The sugar plant by-product residue may contain a filtration residue.

The sugar plant may be chosen from maple tree, birch tree, sugar cane, sugar beet, corn, rice, palm tree, and agave. The sugar plant may be a maple tree.

According to another embodiment, there is provided a method of producing a nutraceutical or cosmeceutical composition comprising a sugar plant syrup-derived product; which comprises the steps of:

a) filtrating a sugar plant syrup;

b) collecting said filtration residue to produce a sugar plant syrup-derived by-product.

The filtration may be effected using a press, centrifugation, cyclonic separation, decantation, extraction, or a mechanical separation process to cause said residue to be free of filtration residue.

The filtration may be effected with a filter containing fine particles, thereby causing said residue to contain filtration residue.

The filter particle may be chosen from diatomaceous earth, celite, kieselguhr, silica, silicon dioxide, calcium, natural sugar sand, ground bones, slop, clay and the like.

The sugar plant may be chosen from the group consisting of maple tree, birch tree, sugar cane, sugar beet, corn, rice, palm tree, and agave. The sugar plant may be a maple tree.

According to another embodiment, there is disclosed a sugar plant residue obtained by the method of the present invention.

The sugar plant may be maple tree.

According to another embodiment, there is provided a nutraceutical or cosmeceutical composition which comprises a sugar plant by-product residue from the purification of sugar plant syrup.

According to another embodiment, there is provided a nutraceutical or cosmeceutical composition which comprises a sugar plant by-product residue according to the present invention.

The sugar plant residue may comprise at least one saccharide chosen from glucose, fructose, galactose, ribose, deoxyribose, mannose, maltose, kojibiose, nigerose, isomaltose, trehalose, β,β-trehalose, α,β-trehalose, sophorose, laminaribiose, gentiobiose, turanose, maltulose, gentiobiulose, mannobiose, melibiose, melibiulose, rutinose, rutinulose, isomaltotriose, nigerotriose, maltotriose, maltotriulose, raffinose, inulin, kestose, nystose, fructosylnystose, bifurcose, a fructooligosaccharide, quebrachitol, arabinogalactan, dextran, inulotriose, and inulotetraose.

The sugar plant residue may comprise at least one nutrient chosen from

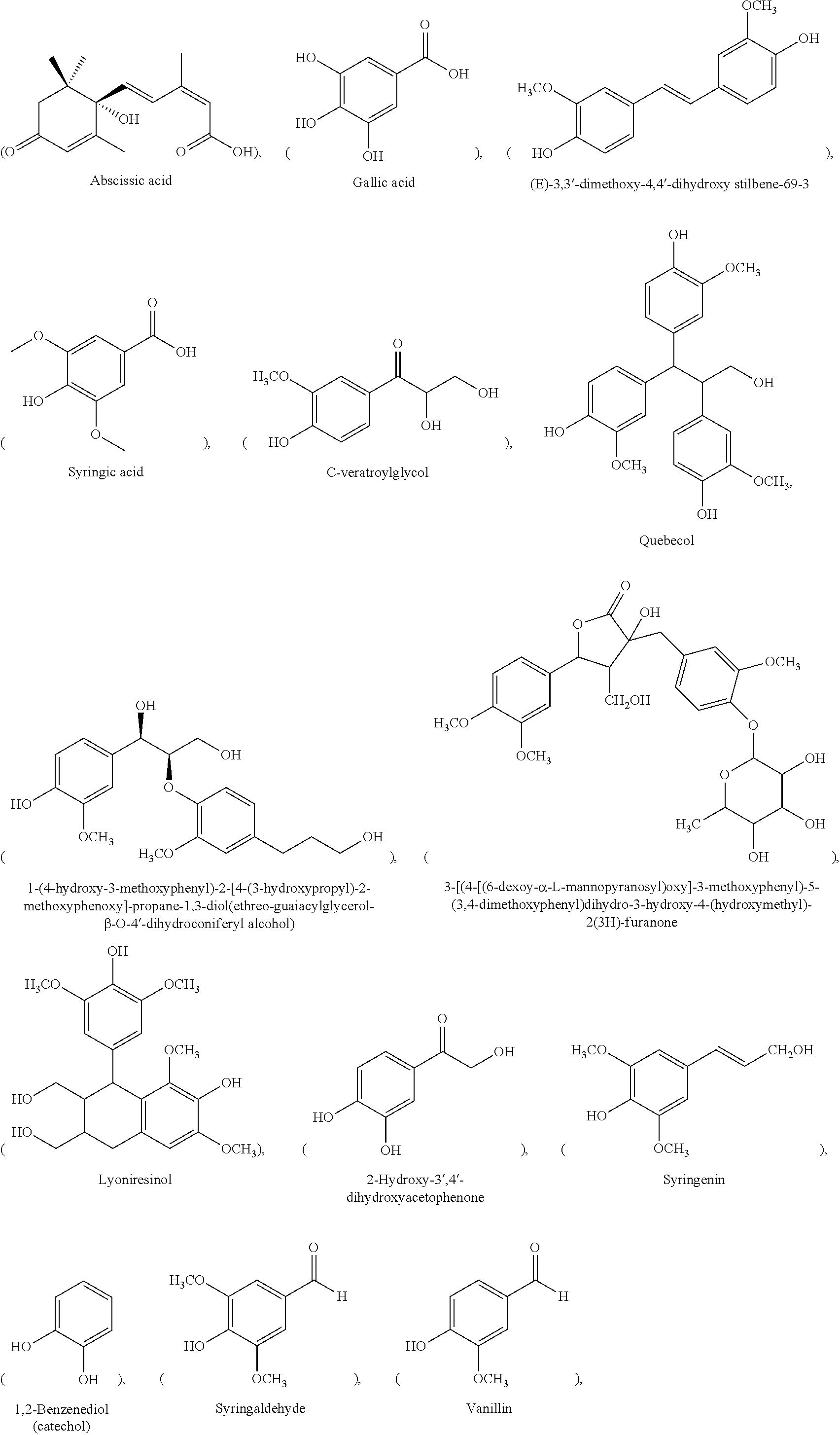

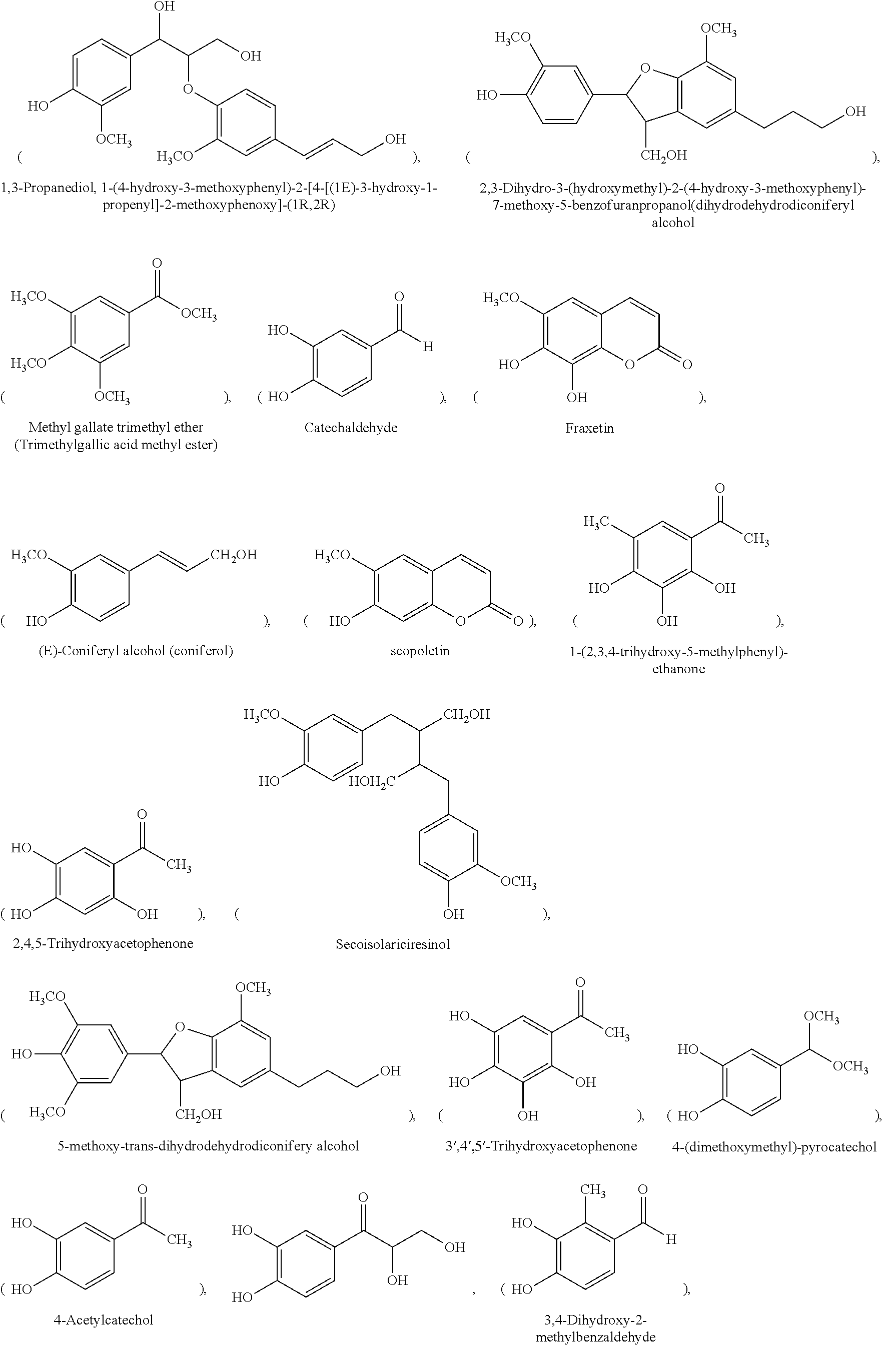

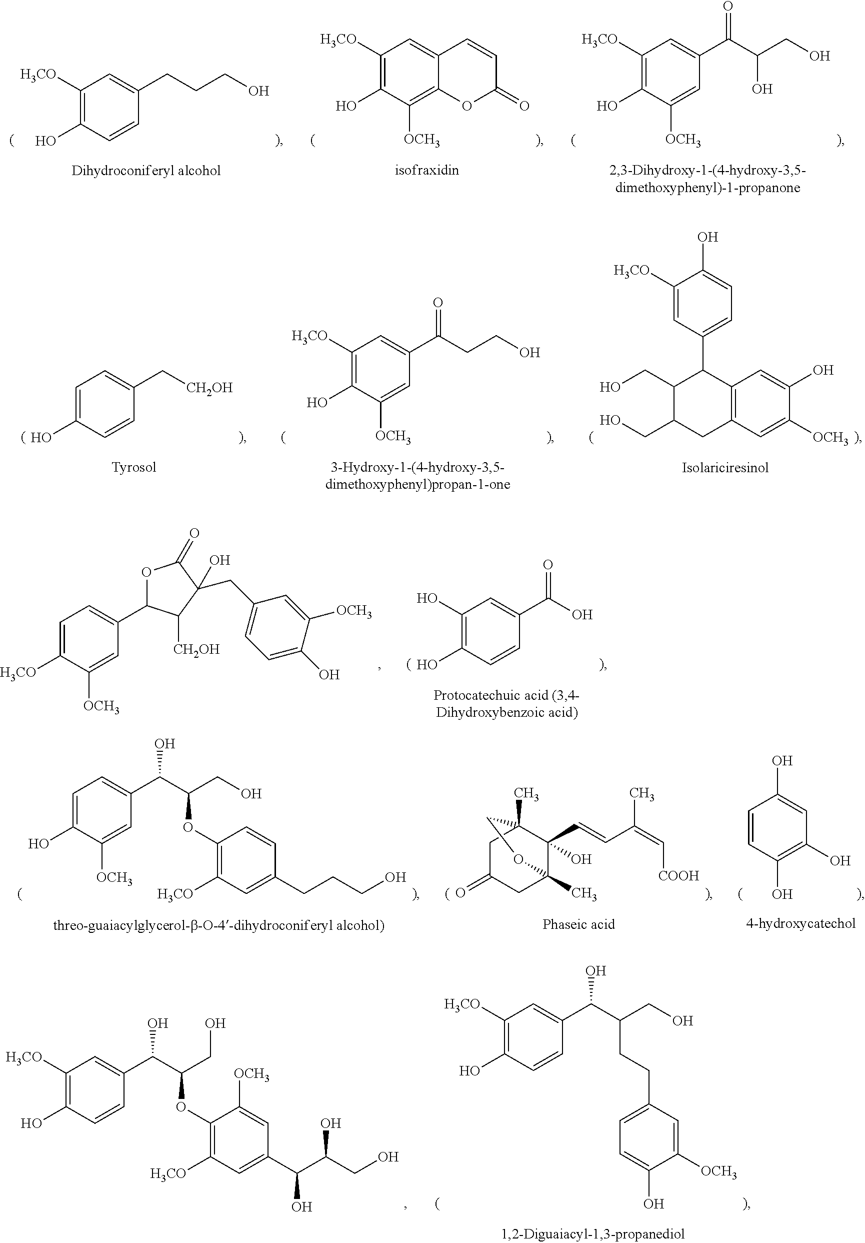

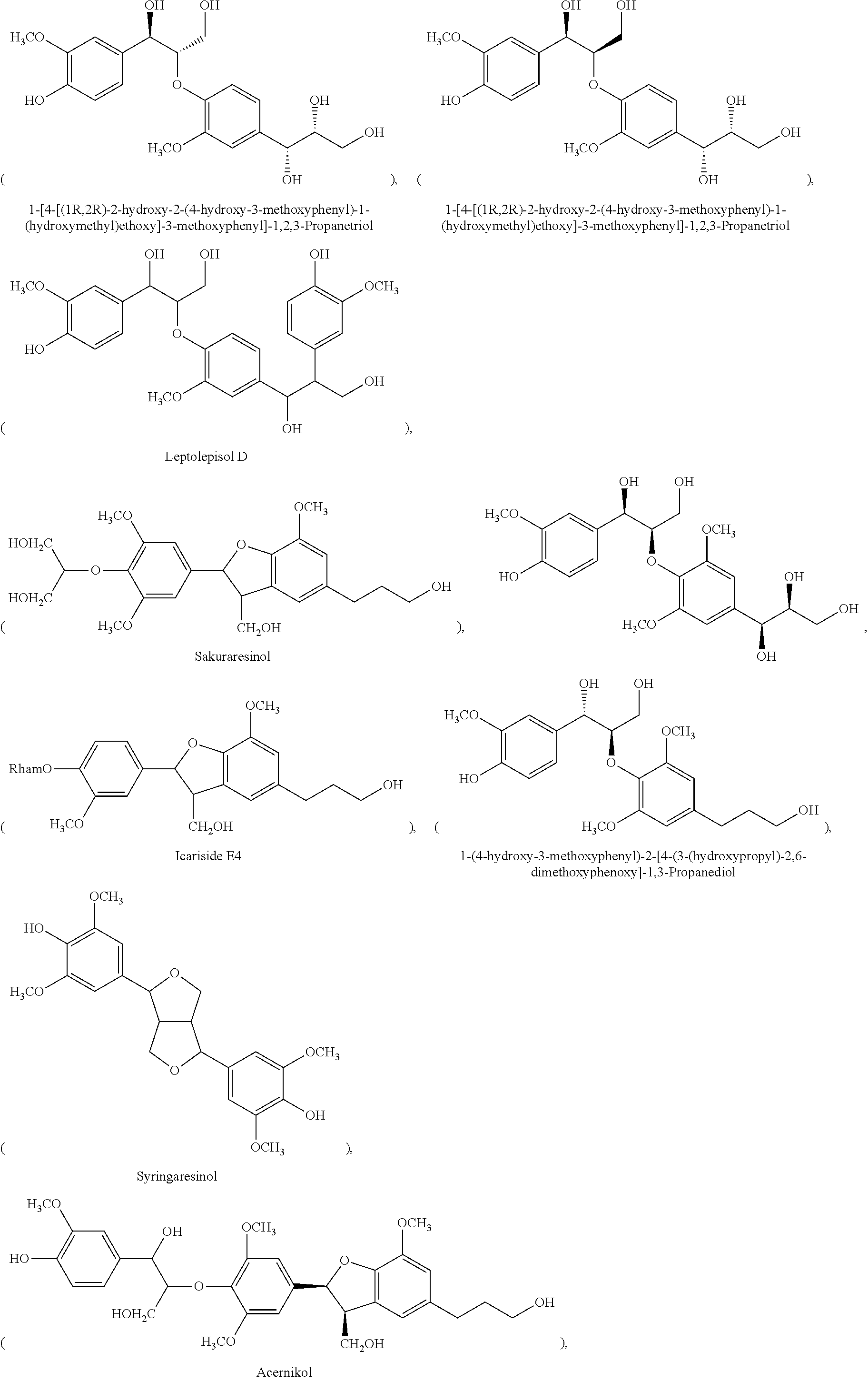

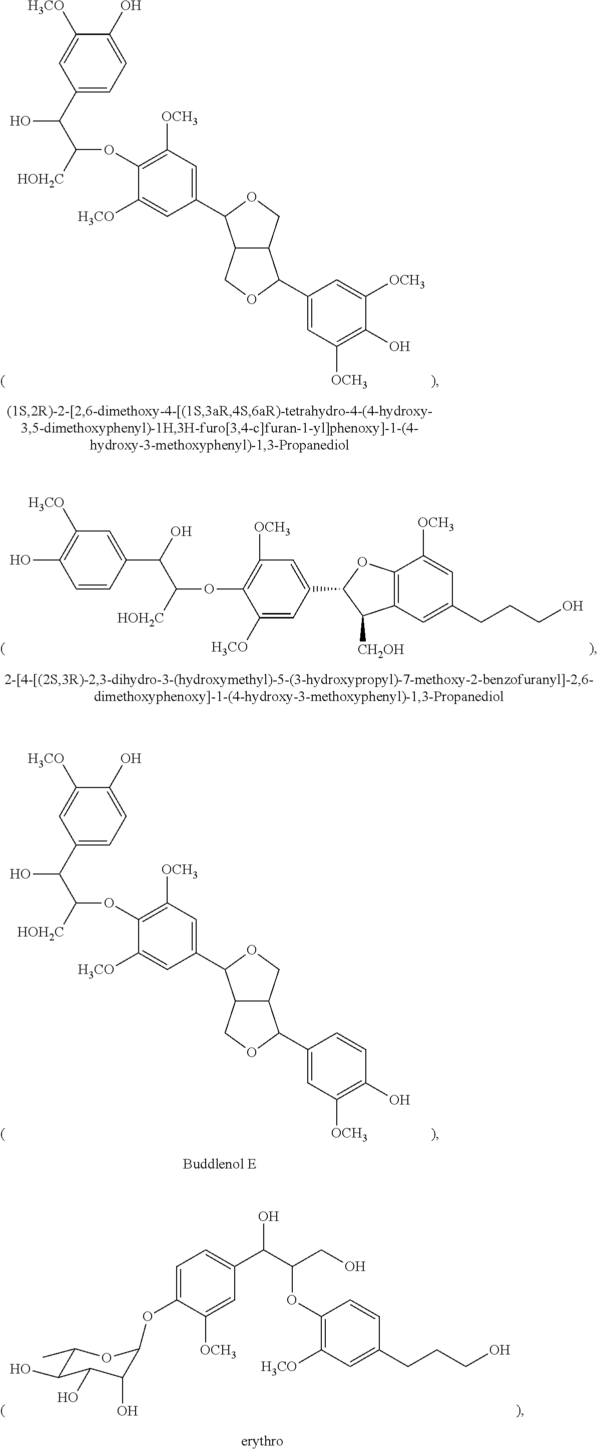 [SMILES: COc1cc(C(O)C(CO)Oc2c(OC)cc(C3OCC4C(c5cc(OC)c(O)c(OC)c5)OCC34)cc2OC)ccc1O ; COc1cc(C(O)C(CO)Oc2c(OC)cc([C@@H]3Oc4c(OC)cc(CCCO)cc4[C@H]3CO)cc2OC)ccc1O ; COc1cc(C(O)C(CO)Oc2c(OC)cc(C3OCC4C(c5ccc(O)c(OC)c5)OCC34)cc2OC)ccc1O ; COc1cc(CCCO)ccc1OC(CO)C(O)c1ccc(OC2OC(C)C(O)C(O)C2O)c(OC)c1]

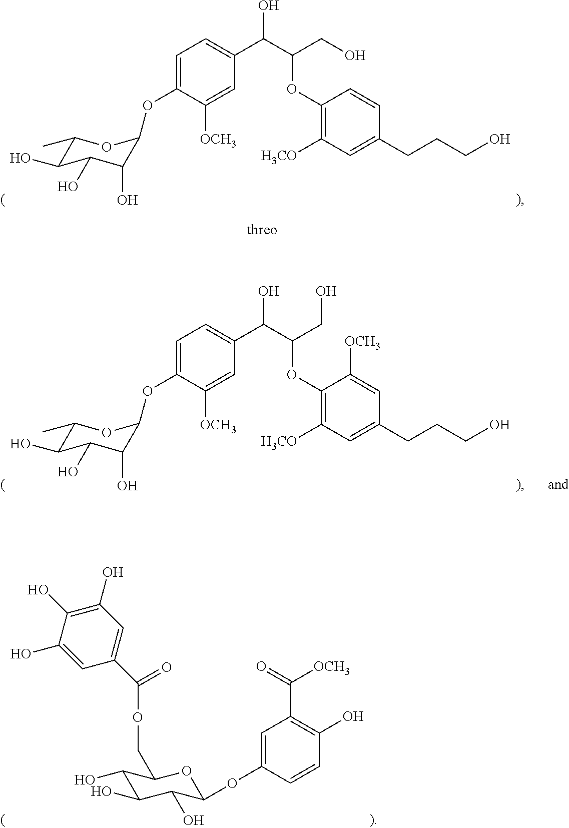 [SMILES: COc1cc(CCCO)ccc1OC(CO)C(O)c1ccc(OC2OC(C)C(O)C(O)C2O)c(OC)c1 ; COc1cc(C(O)C(CO)Oc2c(OC)cc(CCCO)cc2OC)ccc1OC1OC(C)C(O)C(O)C1O ; COC(=O)c1cc(OC2OC(COC(=O)c3cc(O)c(O)c(O)c3)C(O)C(O)C2O)ccc1O]

According to another embodiment, there is provided a nutraceutical or cosmeceutical composition according the present invention for the prophylaxis of an ailment comprising a therapeutically effective amount of a sugar plant by-product residue in association with a pharmaceutically acceptable carrier.

The sugar plant by-product residue may be free of a filtration residue.

The sugar plant by-product residue may contain a filtration residue.

The filtration residue may be chosen from diatomaceous earth, celite, kieselguhr, silica, silicon dioxide, calcium, natural sugar sand, ground bones, slop, clay and the like.

According to another embodiment, there is provided a topical nutraceutical or cosmeceutical composition according to the present invention for improving health condition of skin or various organs comprising a therapeutically effective amount of a sugar plant by-product residue in association with a pharmaceutically acceptable carrier.

The sugar plant by-product residue may be free of a filtration residue.

The sugar plant by-product residue may contain a filtration residue.

The filtration residue may be chosen from diatomaceous earth, celite, kieselguhr, silica, silicon dioxide, calcium, natural sugar sand, ground bones, slop, clay and the like.

According to another embodiment, there is provided a method of prophylaxis of an ailment comprising administering to a subject in need thereof a nutraceutical or cosmeceutical composition according to the present invention.

The ailment may be chosen from a metabolic syndrome, diabetes, a cancer, an arthritis, a micro-organism infection, neurodegenerative disease, an inflammation, an inflammatory diseasecondition, an oxidative stress related disease, an intestinal dysfunction and a heart disease.

The intestinal dysfunction may be chosen from an inflammatory bowel disease, Crohn's disease, an ulcerative colitis, collagenous colitis, lymphocytic colitis, ischaemic colitis, diversion colitis, Behçet's disease, and indeterminate colitis.

The micro-organism infection may be chosen from a bacterial infection, a fungal infection, and combinations thereof.

According to another embodiment, there is provided a use of a nutraceutical or cosmeceutical composition according to the present invention for the prophylaxis of an ailment.

The ailment may be chosen from a metabolic syndrome, diabetes, a cancer, an arthritis, a micro-organism infection, neurodegenerative disease, an inflammation, an inflammatory diseasecondition, an oxidative stress related disease, an intestinal dysfunction and a heart disease.

The intestinal dysfunction may be chosen from an inflammatory bowel disease, Crohn's disease, an ulcerative colitis, collagenous colitis, lymphocytic colitis, ischaemic colitis, diversion colitis, Behçet's disease, and indeterminate colitis.

The micro-organism infection may be chosen from a bacterial infection, a fungal infection, and combinations thereof.

According to another embodiment, there is provided a food sugar replacement with healthy nutritional values, comprising a nutraceutical or cosmeceutical composition according to the present invention.

The food sugar replacement may be a functional food, a food ingredient, an additive, a cosmeto-food, a pharmaceutical, and a food supplement.

According to another embodiment, there is provided a non-food ingredient comprising a nutraceutical or cosmeceutical composition according to the present invention.

The term “sugar plant” is intended to mean any plant used in the production of sugar. Such plants include, without limitation, maple tree, birch tree, sugar cane, sugar beet, corn, rice, palm, and agave among others.

The term “Acer tree” or a “maple tree” is intended to mean a maple tree of a species known to date, such as Acer nigrum, Acer lanum, Acer acuminatum, Acer albopurpurascens, Acer argutum, Acer barbinerve, Acer buergerianum, Acer caesium, Acer campbellii, Acer campestre, Acer capillipes, Acer cappadocicum, Acer carpinifolium, Acer caudatifolium, Acer caudatum, Acer cinnamomifolium, Acer circinatum, Acer cissifolium, Acer crassum, Acer crataegifolium, Acer davidii, Acer decandrum, Acer diabolicum, Acer distylum, Acer divergens, Acer erianthum, Acer erythranthum, Acer fabri, Acer garrettii, Acer glabrum, Acer grandidentatum, Acer griseum, Acer heldreichii, Acer henryi, Acer hyrcanum, Acer ibericum, Acer japonicum, Acer kungshanense, Acer kweilinense, Acer laevigatum, Acer laurinum, Acer lobelii, Acer lucidum, Acer macrophyllum, Acer mandshuricum, Acer maximowiczianum, Acer miaoshanicum, Acer micranthum, Acer miyabei, Acer mono, Acer mono x Acer truncatum, Acer monspessulanum, Acer negundo, Acer ningpoense, Acer nipponicum, Acer oblongum, Acer obtusifolium, Acer oliverianum, Acer opalus, Acer palmatum, Acer paxii, Acer pectinatum, Acer pensylvanicum, Acer pentaphyllum, Acer pentapomicum, Acer pictum, Acer pilosum, Acer platanoides, Acer poliophyllum, Acer pseudoplatanus, Acer pseudosieboldianum, Acer pubinerve, Acer pycnanthum, Acer rubrum, Acer rufinerve, Acer saccharinum, Acer saccharum, Acer sempervirens, Acer shirasawanum, Acer sieboldianum, Acer sinopurpurescens, Acer spicatum, Acer stachyophyllum, Acer sterculiaceum, Acer takesimense, Acer tataricum, Acer tegmentosum, Acer tenuifolium, Acer tetramerum, Acer trautvetteri, Acer triflorum, Acer truncatum, Acer tschonoskii, Acer turcomanicum, Acer ukurunduense, Acer velutinum, Acer wardii, Acer×peronai, Acer×pseudoheldreichii or any new species not yet known.

The term “maple syrup residue” is intended to mean any substance separated from the syrup by any means. These means may by physical, mechanical or chemical, such as centrifugation, cyclonic separation, decantation, filtration, precipitation electrophoresis, evaporation, extraction, and the like.

The term “saccharide” is intended to mean simple sugars or polymers of sugar monomers such as starch and cellulose. It includes monosaccharide, disaccharide, and polysaccharide.

The term “clearing” is intended to mean the act of removing particles from a liquid.

The term “inflammatory condition is intended to mean a condition that results in abnormal inflammation, such as an allergic reaction, a myopathie, an immune disorder, cancer, atherosclerosis, and ischaemic heart disease.

The term “cosmetic” is intended to mean involving or relating to treatment intended to restore or improve a person's appearance.

The term “cosmeceutic” is intended to mean the marriage of cosmetics and pharmaceuticals. Examples of products typically labeled as cosmeceuticals include anti-aging creams and moisturizers. Cosmeceuticals are cosmetic products with biologically active ingredients purporting to have medical or drug-like benefits.

The term “nutraceutical” is intended to mean a food or food product that provides health and medical benefits, including the prevention and treatment of disease.

The term “filtration” is intended to mean the mechanical or physical operation which is used for the separation of solids from fluids (liquids or gases) by interposing a medium through which only the fluid can pass. This includes filtration methods which include but are not limited to filter filtration, microfiltration, ultrafiltration, nanofiltration, reverse osmosis, crossflow filtration, etc.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### BRIEF DESCRIPTION OF THE DRAWINGS

Further features and advantages of the present disclosure will become apparent from the following detailed description, taken in combination with the appended drawings, in which:

FIG. 1 illustrates an expanded assembly diagram of a filter press.

FIG. 2 illustrates a flow diagram for the collection of a sugar plant by-product residue according to one embodiment of the present invention.

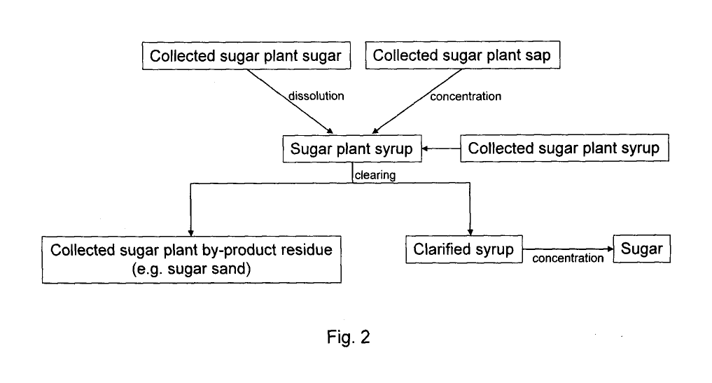

It will be noted that throughout the appended drawings, like features are identified by like reference numerals.

### DETAILED DESCRIPTION OF THE PREFERRED EMBODIMENTS

In embodiments there is disclosed a nutraceutical or cosmeceutical composition. Nutraceuticals are food or food product that provides health and medical benefits, including the prevention and treatment of disease. Products according to the present invention may range from isolated nutrients, dietary supplements, prebiotic, specific diets and herbal products, and processed foods such as cereals, soups, and beverages.

According to another embodiment, the composition of the present invention may be cosmeceuticals products application (exfoliant, masque, onguent, etc) that may be topically applied. The topical composition may be for example, for improving the health condition of skin or various organs.

The composition of the present invention may be consumed for the prophylaxis of ailments such as diabetes, such as diabetes melitus, cancer, arthritis, micro-organism infection such as bacterial infections or fungal infections, neurodegenerative diseases, inflammatory diseases, an inflammatory condition, oxidative stress related diseases, intestinal dysfunction, and heart diseases. The composition of the present invention may also be used as a functional food, a food ingredient, an additive, a non-food ingredient, a cosmeto-food, a pharmaceutical, and a food supplement.

The sugar plant by-product residue may be obtained from any sugar plant syrup. This includes for example any type of maple syrup, proper for consumption as food, but also non-conform syrups that are obtained late in the maple syrup production season, and which have defects (e.g. undesired wooden or moldy taste). There are many methods to filter maple syrup and collect maple tree syrup filtration residue. Below is enumerated some illustrative manner of maple syrup filtration.

Syrup filtration residue may be used as a natural filter membrane in the maple syrup production or a continuous filtration mode or by being frozen and then thaw prior its reuse.

a) Filtering Maple Syrup with a Cloth Filter

When sap is boiled, the minerals and the others nutrients such as polyphenol, complex sugar, amino acids, organic acids, vitamins, that are naturally present in the sap are concentrated or hardened into a substance called niter, commonly known as sugar sand

The filtering process allows the removal of the sugar sand.

a.i) Filter the Syrup Hot

The first rule in filtering is that the syrup must be hot. There are no shortcuts around this. It is best to filter syrup that has just been drawn off from the evaporator or finisher. As the syrup cools it begins to thicken and the filtering process is all but impossible.

The second rule of filtering: patience. With the obvious exception of the filter press, the filtering process is not a task that is quickly completed. In fact, if the syrup runs quickly through the filter, one should be concerned. The syrup must work its way through and around the dense fibers of the filter fabric. If it can run through quickly, it is a sure sign that the filter fibers have been broken down and the filter is not working properly. It may be time to get a new filter.

A good filtration takes time, but time means cooling syrup. Filtering and canning units, built to keep the syrup clean and hot during the filtering process, help resolve this problem. If one does not have a commercial filter tank, one should design a system that puts the filter inside a covered container. It also helps to filter larger batches so there is more mass to the hot syrup. Pour in hot syrup, cover it to keep the heat inside, and be patient as the syrup oozes through the filter. Keep a thermometer near by; if the syrup cools below 180° F. (about 82° C.) during the filtering process, heat the syrup to the recommended 180° F. to 200° F. (about 82° C. to 93° C.) canning range. Care must be taken so that the syrup is not overheated, however. Syrup heated beyond the 200 ° F. (93° C.) mark is likely to produce more sugar sand and may need to be re-filtered.

a.ii) Layer Pre-Filters Inside Final Filters

A two-layer filter system may be used. According to one embodiment, one may place a pre-filter inside (or on top of) the final filter. A pre-filter is a thin fabric made of rayon cellulose, often referred to as “paper filters”. The purpose of the pre-filter is to give an extra layer of filtering material and to keep the largest particles away from the “clean” final filter. This lets the final filter do a better job and reduces the number of cleanings required. The thinner, less expensive and easier to clean pre-filters can be changed easily and more often while the final filter is kept cleaner and can be used over a longer period. The bulk of the sugar sand should be caught in the pre-filter. Use the filters together, not as a 2-step process, to minimize heat loss. Don't be too concerned about built up sugar sand. If syrup is still moving through the filters, leave them alone; the sugar sand is a great filter bed. It is possible to recuperate a portion of this precipitate to be used as a natural filter during filtration of the syrup.

a.iii) Cone Vs. Flat/Synthetic Vs. Wool

The decision to buy Cone-shaped or flat filters will be determined by the filtering equipment. Cone filters are used in tall, covered filter tanks with hooks at the top to hold the filter “tabs.” Flat filters are used in filter and canning units with flat wire racks, or in homemade colander/sieve operations. Flat filters are only available in synthetic fabrics. If one is using cones, one can choose from synthetic or wool, as a matter of personal preference. The old-time traditional filter is made of wool; many still refer to them as a “hat” because they look like an old wool hat when turned upside down. Development of superior synthetic fibers over the past few decades, however, have led the vast majority of sugarmakers to switch to easy care synthetic filters, which lack the shrinkage and moth problems associated with wool.

a.iv) Preparing and Cleaning the Filter

Soaking the filter in hot water for a few minutes is recommended when using for the first time. This ensures that the filters are clean and that they will not contaminate new syrup with old bacteria. The filter works a little better if dampened slightly in hot water before use. With proper care, the filters can be used many times, even years. It is recommended that one rinse the filters by turning them inside out and flushing them with hot water until the water runs clean. One should never use soaps or detergents, which can flavor the syrup. Handle the filters carefully when cleaning; never twist, wring or stretch the filters, but gently squeeze the water out.

b) Filtering Maple Syrup with a Filter Press

A filter press includes filter plates which are constructed of light weight aluminum for low maintenance and fast heating. The filter allows thorough and rapid filtering, which allows the collection of the syrup residue.

b.i) Principles of Operation

The pressure filter uses a special paper or washable fabric such as “Orlon™”, which traps the sand particles, and a filter aid powder, which is added to the liquid before going through the press to hold the trapped sand in suspension.

In embodiments, silica powder, ground bones, slop, clay, clay dust and diatomaceous earth are acceptable for use in the filter presses as a filter aid powder.

According to an embodiment, it is defined that any suitable mechanical process that can result in a mechanical separation can be used. By example, centrifugation, cyclonic separation, decantation, extraction, pressing, sedimentation, sieving, and the like are mechanical separation process which can be used. Moreover, it can be noted that a filtration process used can be centrifugation. Chemical processes such as precipitation may also be employed, for example, by modifiying the pH of the syrup being treated.

According to another embodiment, the filter aid must be used in the process or the papers will plug immediately. Hot syrup is pumped into the interior of the plates and frames of the press. The flow continues through the filter paper and exits the press. The dirt, sugar sand and filter aid powder are collected against the filter paper inside the frame.

b.ii) The Operation of Filtering Syrup

Before the liquid is filtered, some preparation is necessary.

b.iii) Assemble the Press:

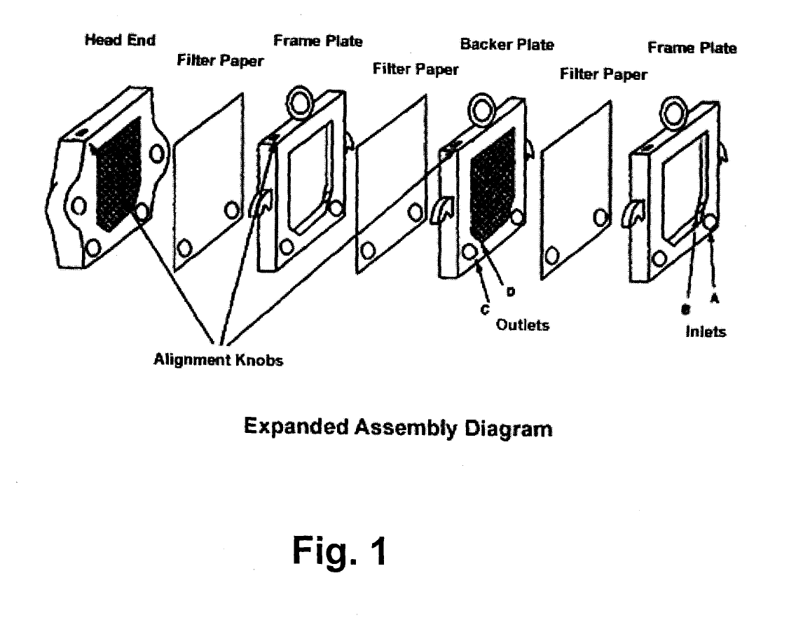

The operating of a filter press is very straightforward. A single stage of the filter consists of a sheet of filter paper sandwiched between a frame plate and a backer plate. (FIG. 1) Hot syrup is pumped into the interior of the frame plate through holes A and B. The flow continues through the filter paper, onto the backer plate grid, and exits to hole D of the backer plate via the smaller holes C. The filtered dirt collects against the filter paper inside the frame plate. Each plate and frame fills one after the other in succession.

Because of the previously described flow path, proper assembly of the filter press is crucial for alignment of the syrup flow holes and correct operation of the press. FIG. 1 shows an expanded assembly diagram. Starting at the fixed head end, the press is assembled as follows:

Place a filter paper over the head, lining up the holes.

With locating knobs on left (facing the head block) install frame plate.

Place a filter paper over frame plate.

With locating knobs on left, install backer plate.

Repeat steps 2 through 4 for the next 6 stages.

Install final filter paper.

Install end block.

Place washers on the threaded rods, screw on wing nuts and hand tighten. Note that the paper filters also act as gaskets, so some seepage will normally occur during operation.

Note: When assembling the filter press be careful to properly align the plate locating knobs to the left (facing the head block). The reversal of only one element can adversely affect the flow pattern and result in partially filtered, cloudy syrup. There are three types of filtration: conical gravitational filter (19%), circular filter press (14%) and plate-and-frame filter press (43%).

b.iv) Heat the Syrup:

The syrup must be heated to approximately 195° F. (about 95.5° C.). Cold liquid cannot be filtered.

Next, an amount of filter aid (for example, diatomaceous earth) may be be mixed into the hot liquid. Filter aid imparts no taste and is completely filtered out of the liquid. The function of the filter aid is to act as an additional filtering layer as it accumulates on the filter papers allowing more clean syrup to pass through the papers. The amount of filter aid used depends primarily on grade of syrup, the amount of suspended solids, the temperature of the syrup and the volume of syrup. A general guideline is to use 1 to 3 cups of filter aid powder for every 5 gallons of liquid. This amount can be adjusted to suit the application, adding more for syrup with more suspended solids and reducing the amount for cleaner syrup.

The filter aid should be added near the outlet of the finishing pan and stirred completely. Add the filter aid slowly and continue to mix until the filter press is started. (The filter aid powder can settle out if not stirred.)

b.v) Start the Press

Following syrup preparation, the press is ready for operation. If the press is equipped with an electrically driven gear pump, make sure the pump power switch is off and plug the power cord into a 110 VAC grounded outlet.

Place the inlet hose into the syrup to be filtered and the outlet hose into a suitable container to receive the filtered syrup. To assure the pump properly primes, place the pump at or below the level of the syrup pan.

Start the pump and allow the syrup to enter the filter.

If the filter is equipped with an air driven diaphragm type pump, the pump is started by slightly cracking the pressure regulator to start, and slowly increasing the pressure to the operating pumping rate. When the syrup appears at the outlet, stop the pump for about a minute to allow the press plates to heat up. Restart the pump and continue until the holding pan is empty, stirring the liquid as needed. One may continue filtering until the pressure gauge reads approximately 50 pounds. At this point the frames will be full or the papers will be plugged. The press must be cleaned and new papers installed in order to filter the next batch. (Note: the press must also be cleaned for re-use if the frames become too cool before the next batch of syrup is ready to be filtered.)

Run ½ gallon hot water through the filter press before disassembly to flush the pump of syrup. (This amount of water is approximate and more or less may be used.) This sweet water may be saved and added when finishing the next batch of syrup.

b.vi) Disassembly and Cleaning

The press is disassembled for cleaning between batches by loosening the wing nuts enough to allow free movement of the plates and frames. The syrup in the press will drain into the drip pan and can be filtered with the next batch.

The frames can be lifted out and cleaned with hot water only.

The filter papers should be discarded and new ones used for each new batch.

c) Sugar Sand or Sugar Plant By-Product Residue

The sugar plant by-product residue (e.g. sugar sand) may contain a wide number of nutrients such as abscisic acid, phaseic acid, and other compounds according to Table 1 below:

TABLE 1
Phenolic compounds identified in sugar plant syrup

| | Chemical name | Chemical structure | Properties |
|---|---|---|---|
| 1 | Gallic acid | 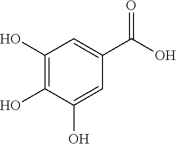 [SMILES: O=C(O)c1cc(O)c(O)c(O)c1] | antioxidative |
| 2 | (E)-3,3′-dimethoxy-4,4′- dihydroxy stilbene-69-3 | 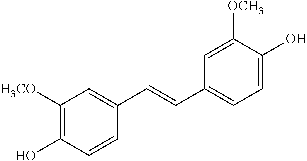 [SMILES: COc1cc(/C=C/c2ccc(O)c(OC)c2)ccc1O] | antioxidative |
| 3 | Syringic acid | 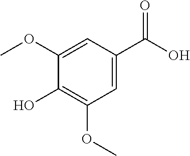 | antioxidative |
| 4 | C-veratroylglycol | 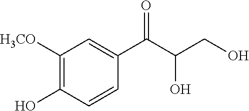 [SMILES: COc1cc(C(=O)C(O)CO)ccc1O] | No assay tested |
| 5 | Quebecol (New) | 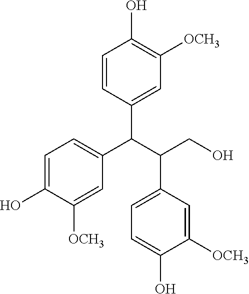 [SMILES: COc1cc(C(CO)C(c2ccc(O)c(OC)c2)c2ccc(O)c(OC)c2)ccc1O] | |
| 6 | 1-(4-hydroxy-3- methoxyphenyl)-2-[4-(3- hydroxypropyl)-2- methoxyphenoxy]- propane-1,3-diol (ethreo-guaiacylglycerol- β-O-4′-dihydroconiferyl alcohol) | 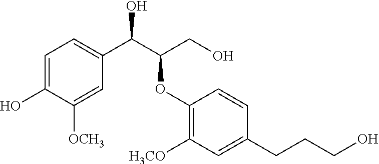 [SMILES: COc1cc([C@@H](O)[C@@H](CO)Oc2ccc(CCCO)cc2OC)ccc1O] | Antioxidative |
| 7 | 3-[(4-[(6-dexoy-α-L- mannopyranosyl)oxy]-3- methoxyphenyl)-5-(3,4- dimethoxyphenyl)dihydro- 3-hydroxy-4- (hydroxymethyl)-2(3H)- furanone | 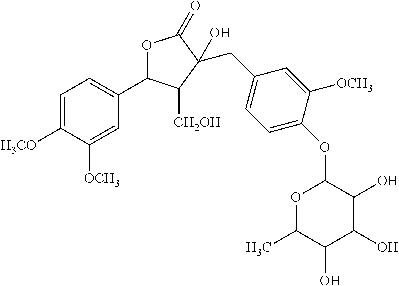 [SMILES: COc1ccc(C2OC(=O)C(O)(Cc3ccc(OC4OC(C)C(O)C(O)C4O)c(OC)c3)C2CO)cc1OC] | α- glucosidase- inhibiting, SOD reactive oxygen radical- scavenging, and human HL-60 proliferation- inhibiting actions |
| 8 | Lyoniresinol | 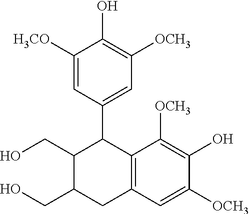 [SMILES: COc1cc(C2c3c(cc(OC)c(O)c3OC)CC(CO)C2CO)cc(OC)c1O] | Antioxidant/ tyrosinase inhibitory |
| 9 | 2-Hydroxy-3′,4′- dihydroxyacetophenone | 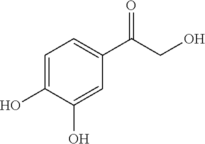 [SMILES: O=C(CO)c1ccc(O)c(O)c1] | antioxidant |
| 10 | Syringenin | 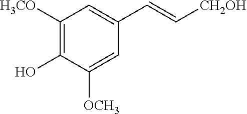 [SMILES: COc1cc(/C=C/CO)cc(OC)c1O] | TNFα- secretion inhibitor |
| 11 | 1,2-Benzenediol(catechol) | 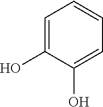 | inhibit the activity of tyrosinase and inhibit the synthesis of melanin/Anti- oxidant/ Antibacterial |
| 12 | Syringaldehyde | 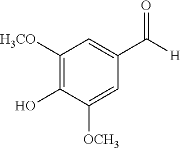 [SMILES: COc1cc(C=O)cc(OC)c1O] | antioxidant |
| 13 | Vanillin | 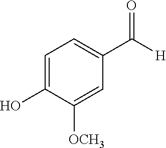 [SMILES: COc1cc(C=O)ccc1O] | antioxidant |
| 14 | 1,3-Propanediol, 1-(4- hydroxy-3- methoxyphenyl)-2-[4- [(1E)-3-hydroxy-1- propenyl]-2- methoxyphenoxy]-, (1R,2R)- | 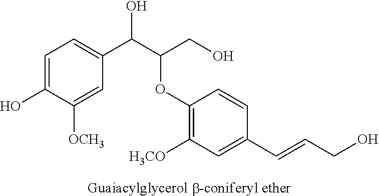 [SMILES: COc1cc(C(O)C(CO)Oc2ccc(/C=C/CO)cc2OC)ccc1O] | |
| 15 | 2,3-Dihydro-3- (hydroxymethyl)-2-(4- hydroxy-3- methoxyphenyl)-7- methoxy-5- benzofuranpropanol (dihydrodehydrodiconiferyl alcohol | 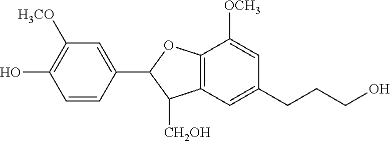 [SMILES: COc1cc(C2Oc3c(OC)cc(CCCO)cc3C2CO)ccc1O] | Antitumor/ |
| 16 | Methyl gallate trimethyl ether (Trimethylgallic acid methyl ester) | 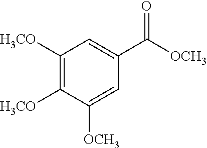 [SMILES: COC(=O)c1cc(OC)c(OC)c(OC)c1] | |
| 17 | Catechaldehyde | 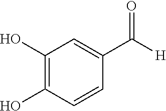 | |
| 18 | Fraxetin | 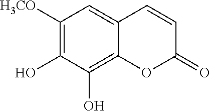 [SMILES: COc1cc2ccc(=O)oc2c(O)c1O] | antioxidant |
| 19 | (E)-Coniferyl alcohol (coniferol) | 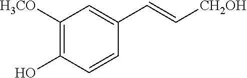 [SMILES: COc1cc(/C=C/CO)ccc1O] | |
| 20 | scopoletin | 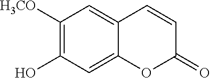 [SMILES: COc1cc2ccc(=O)oc2cc1O] | |
| 21 | 1-(2,3,4-trihydroxy-5- methylphenyl)-ethanone | 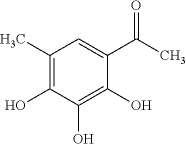 | |
| 22 | 2,4,5- Trihydroxyacetophenone | 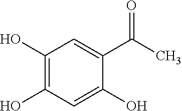 | |
| 23 | Secoisolariciresinol | 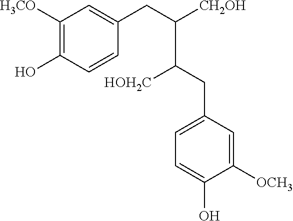 [SMILES: COc1cc(CC(CO)C(CO)Cc2ccc(O)c(OC)c2)ccc1O] | antioxidant |
| 24 | 5-methoxy-trans- dihydrodehydrodiconifery alcohol | 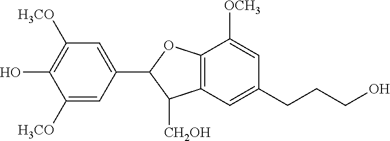 [SMILES: COc1cc(C2Oc3c(OC)cc(CCCO)cc3C2CO)cc(OC)c1O] | |
| 25 | 3′,4′,5′- Trihydroxyacetophenone | 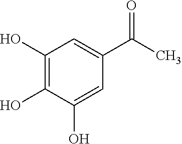 | |
| 26 | 4-(dimethoxymethyl)- pyrocatechol | 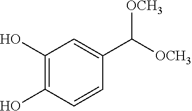 [SMILES: COC(OC)c1ccc(O)c(O)c1] | |
| 27 | 4-Acetylcatechol | 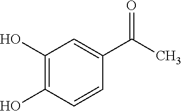 | |
| 28 | new | 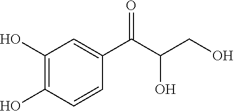 [SMILES: O=C(c1ccc(O)c(O)c1)C(O)CO] | |
| 29 | 3,4-Dihydroxy-2- methylbenzaldehyde | 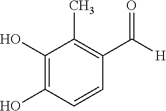 | |
| 30 | Dihydroconiferyl alcohol | 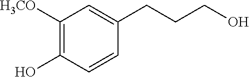 [SMILES: COc1cc(CCCO)ccc1O] | |
| 31 | isofraxidin | 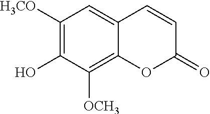 [SMILES: COc1cc2ccc(=O)oc2c(OC)c1O] | |
| 32 | 2,3-Dihydroxy-1-(4- hydroxy-3,5- dimethoxyphenyl)-1- propanone | 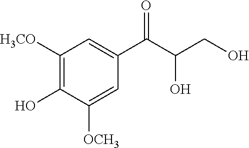 [SMILES: COc1cc(C(=O)C(O)CO)cc(OC)c1O] | |
| 33 | Tyrosol | 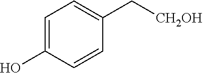 | |
| 34 | 3-Hydroxy-1-(4-hydroxy- 3,5- dimethoxyphenyl)propan- 1-one | 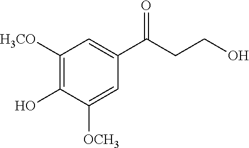 [SMILES: COc1cc(C(=O)CCO)cc(OC)c1O] | |
| 35 | Isolariciresinol | 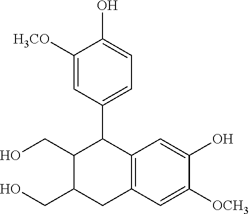 [SMILES: COc1cc(C2c3cc(O)c(OC)cc3CC(CO)C2CO)ccc1O] | |
| 36 | new | 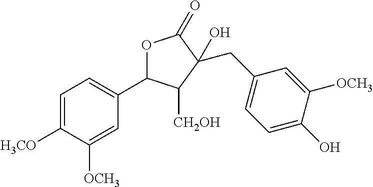 [SMILES: COc1cc(CC2(O)C(=O)OC(c3ccc(OC)c(OC)c3)C2CO)ccc1O] | |
| 37 | Protocatechuic acid (3,4- Dihydroxybenzoic acid) | 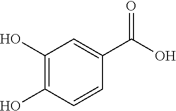 [SMILES: O=C(O)c1ccc(O)c(O)c1] | |
| 38 | threo-guaiacylglycerol-β- O-4′-dihydroconiferyl alcohol | 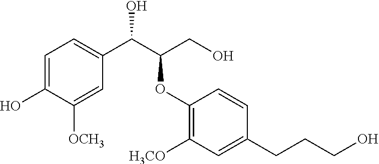 [SMILES: COc1cc([C@H](O)[C@@H](CO)Oc2ccc(CCCO)cc2OC)ccc1O] | |
| 39 | Phaseic acid | 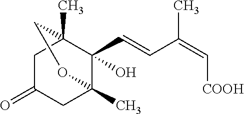 | |
| 40 | 4-hydroxycatechol | 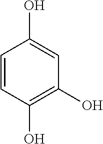 | |
| 41 | new | 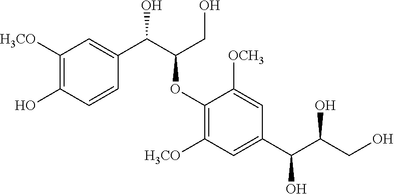 [SMILES: COc1cc([C@H](O)[C@@H](CO)Oc2c(OC)cc([C@H](O)[C@@H](O)CO)cc2OC)ccc1O] | |
| 42 | 1,2-Diguaiacyl-1,3- propanediol | 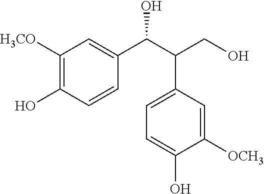 [SMILES: COc1cc(C(CO)[C@@H](O)c2ccc(O)c(OC)c2)ccc1O] | |
| 43 | 1-[4-[(1R,2R)-2-hydroxy- 2-(4-hydroxy-3- methoxyphenyl)-1- (hydroxymethyl)ethoxy]-3- methoxyphenyl]-1,2,3- Propanetriol (one chain is ethreo, and other one is threo) | 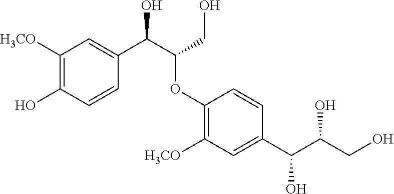 [SMILES: COc1cc([C@@H](O)[C@H](CO)Oc2ccc([C@@H](O)[C@H](O)CO)cc2OC)ccc1O] | |
| 44 | 1-[4-[(1R,2R)-2-hydroxy- 2-(4-hydroxy-3- methoxyphenyl)-1- (hydroxymethyl)ethoxy]-3- methoxyphenyl]-1,2,3- Propanetriol (both chain are threo) | 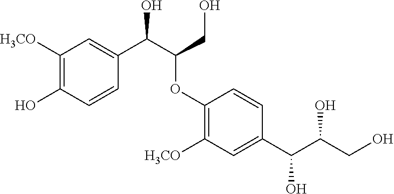 [SMILES: COc1cc([C@@H](O)[C@@H](CO)Oc2ccc([C@@H](O)[C@H](O)CO)cc2OC)ccc1O] | |
| 45 | Leptolepisol D | 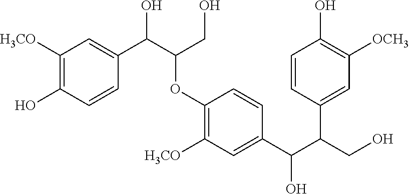 [SMILES: COc1cc(C(O)C(CO)Oc2ccc(C(O)C(CO)c3ccc(O)c(OC)c3)cc2OC)ccc1O] | |
| 46 | Sakuraresinol | 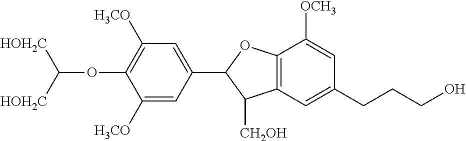 [SMILES: COc1cc(C2Oc3c(OC)cc(CCCO)cc3C2CO)cc(OC)c1OC(CO)CO] | |
| 47 | New (isomer of LL/VIII/154C) | 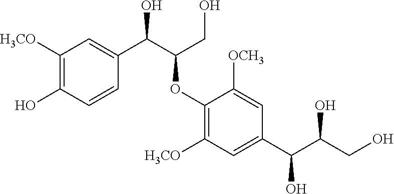 [SMILES: COc1cc([C@@H](O)[C@@H](CO)Oc2c(OC)cc([C@H](O)[C@@H](O)CO)cc2OC)ccc1O] | |
| 48 | Icariside E4 | 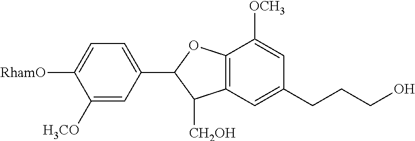 | |
| 49 | | Waiting for NMR | |
| 50 | 1-(4-hydroxy-3- methoxyphenyl)-2-[4-(3- hydroxypropyl)-2,6- dimethoxyphenoxy]-1,3- Propanediol | 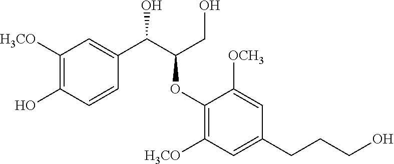 [SMILES: COc1cc([C@H](O)[C@@H](CO)Oc2c(OC)cc(CCCO)cc2OC)ccc1O] | |
| 51 | Syringaresinol | 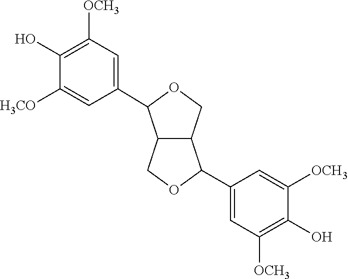 [SMILES: COc1cc(C2OCC3C(c4cc(OC)c(O)c(OC)c4)OCC23)cc(OC)c1O] | |
| 52 | Acernikol | 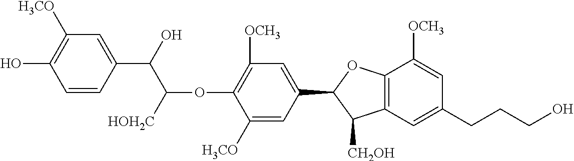 [SMILES: COc1cc(C(O)C(CO)Oc2c(OC)cc([C@H]3Oc4c(OC)cc(CCCO)cc4[C@H]3CO)cc2OC)ccc1O] | |
| 53 | (1S,2R)-2-[2,6- dimethoxy-4- [(1S,3aR,4S,6aR)- tetrahydro-4-(4-hydroxy- 3,5-dimethoxyphenyl)- 1H,3H-furo[3,4-c]furan-1- yl]phenoxy]-1-(4-hydroxy- 3-methoxyphenyl)-1,3- Propanediol | 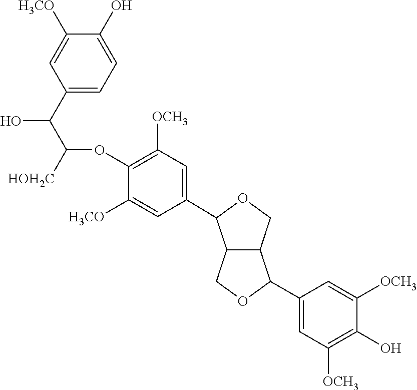 [SMILES: COc1cc(C(O)C(CO)Oc2c(OC)cc(C3OCC4C(c5cc(OC)c(O)c(OC)c5)OCC34)cc2OC)ccc1O] | |
| 54 | 2-[4-[(2S,3R)-2,3-dihydro- 3-(hydroxymethyl)-5-(3- hydroxypropyl)-7- methoxy-2-benzofuranyl]- 2,6-dimethoxyphenoxy]-1- (4-hydroxy-3- methoxyphenyl)-1,3- Propanediol | 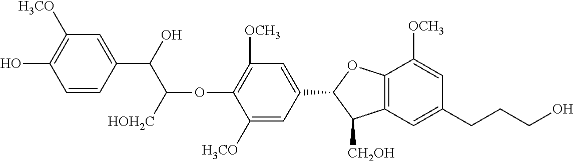 [SMILES: COc1cc(C(O)C(CO)Oc2c(OC)cc([C@@H]3Oc4c(OC)cc(CCCO)cc4[C@H]3CO)cc2OC)ccc1O] | |
| 55 | Buddienol E | 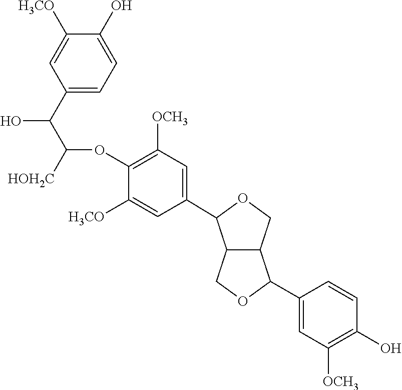 [SMILES: COc1cc(C(O)C(CO)Oc2c(OC)cc(C3OCC4C(c5ccc(O)c(OC)c5)OCC34)cc2OC)ccc1O] | |
| 56 | | 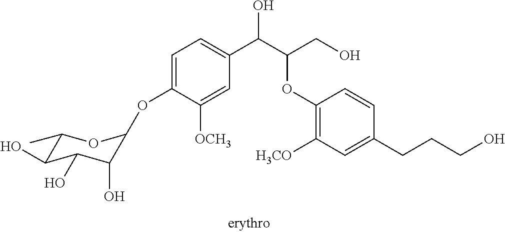 [SMILES: COc1cc(CCCO)ccc1OC(CO)C(O)c1ccc(OC2OC(C)C(O)C(O)C2O)c(OC)c1] | |
| 57 | | 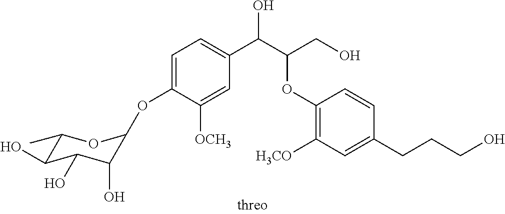 [SMILES: COc1cc(CCCO)ccc1OC(CO)C(O)c1ccc(OC2OC(C)C(O)C(O)C2O)c(OC)c1] | |
| 58 | | 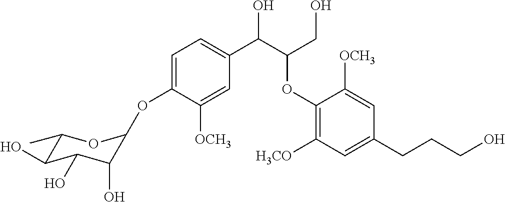 [SMILES: COc1cc(C(O)C(CO)Oc2c(OC)cc(CCCO)cc2OC)ccc1OC1OC(C)C(O)C(O)C1O] | |
| 59 | | 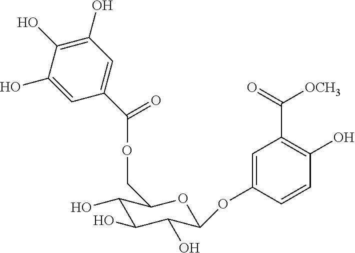 [SMILES: COC(=O)c1cc(OC2OC(COC(=O)c3cc(O)c(O)c(O)c3)C(O)C(O)C2O)ccc1O] | |

According to another embodiment, the sugar plant by-product residue may also contain saccharides, such as mono saccharides, disaccharides, trisaccharides, oligosaccharides, and polysaccharides, which include but are not limited to glucose, fructose, galactose, ribose, deoxyribose, mannose, maltose, kojibiose, nigerose, isomaltose, trehalose, β,β-trehalose, α,β-trehalose, sophorose, laminaribiose, gentiobiose, turanose, maltulose, gentiobiulose, mannobiose, melibiose, melibiulose, rutinose, rutinulose, isomaltotriose, nigerotriose, maltotriose, maltotriulose, raffinose, inulin, kestose, nystose, fructosylnystose, bifurcose, a fructooligosaccharide, quebrachitol, arabinogalactan, dextran, inulotriose, inulotetraose.

According to some embodiments, in use the sugar plant by-product residue may be included in nutraceutical or cosmeceutical compositions. Alternatively, the sugar plant by-product residues may be used for any other suitable applications, such as, in the case of sugar sand, in applications requiring fine granular materials, (e.g. as an abrasive, as a filter material, and the likes).

While preferred embodiments have been described above and illustrated in the accompanying drawings, it will be evident to those skilled in the art that modifications may be made without departing from this disclosure. Such modifications are considered as possible variants comprised in the scope of the disclosure.

1. A method of producing a nutraceutical or cosmeceutical composition comprising a sugar plant by-product residue comprising the step of:
a) collecting a sugar plant by-product residue from the clearing during the production of sugar or syrup to produce a syrup-derived by-product residue.

2. The method of claim 1, wherein collection of said sugar plant by-product residue is by filtration, distillation, pressing, sedimentation, sieving, precipitation or centrifugation of sugar plant syrup.

3. The method of claim 2, wherein said filtration is effected using a filter containing one of diatomaceous earth, celite, kieselguhr, silica, silicon dioxide, calcium, natural sugar sand, ground bones, slop, clay and the like.

4. The method of claim 3, wherein when said sugar plant by-product residue contains a filtration residue.

5. The method of any one of claims 1 to 4, wherein said sugar plant is chosen from maple tree, birch tree, sugar cane, sugar beet, corn, rice, palm tree, and agave.

6. The method of claims 1 to 5, wherein said sugar plant is a maple tree.

7. A method of producing a nutraceutical or cosmeceutical composition comprising a sugar plant syrup-derived product; which comprises the steps of:
a) filtrating a sugar plant syrup;
b) collecting said filtration residue to produce a sugar plant syrup-derived by-product.

8. The method of claim 7, wherein said filtration is effected using a press, centrifugation, cyclonic separation, decantation, extraction, or a mechanical separation process to cause said residue to be free of filtration residue.

9. The method of claim 7, wherein said filtration is effected with a filter containing fine particles, thereby causing said residue to contain filtration residue.

10. The method of claim 9, wherein said filter particle is chosen from diatomaceous earth, celite, kieselguhr, silica, silicon dioxide, calcium, natural sugar sand, ground bones, slop, clay and the like.

11. The method of any one of claims 7 to 10, wherein said sugar plant is chosen from the group consisting of maple tree, birch tree, sugar cane, sugar beet, corn, rice, palm tree, and agave.

12. The method of any one of claims 7 to 10, wherein said sugar plant is a maple tree.

13. A sugar plant residue obtained by the method of any one of claims 1 to 12.

14. The sugar plant residue of claim 13, wherein said sugar plant is maple tree.

15. A nutraceutical or cosmeceutical composition which comprises a sugar plant by-product residue from the purification of sugar plant syrup.

16. A nutraceutical or cosmeceutical composition which comprises a sugar plant by-product residue according to any one of claim 13 or 14.

17. The nutraceutical or cosmeceutical composition as claimed in any one of claim 15 or 16, wherein said sugar plant residue comprises at least one saccharide chosen from glucose, fructose, galactose, ribose, deoxyribose, mannose, maltose, kojibiose, nigerose, isomaltose, trehalose, β,β-trehalose, α,β-trehalose, sophorose, laminaribiose, gentiobiose, turanose, maltulose, gentiobiulose, mannobiose, melibiose, melibiulose, rutinose, rutinulose, isomaltotriose, nigerotriose, maltotriose, maltotriulose, raffinose, inulin, kestose, nystose, fructosylnystose, bifurcose, a fructooligosaccharide, quebrachitol, arabinogalactan, dextran, inulotriose, and inulotetraose.

18. The nutraceutical or cosmeceutical composition as claimed in any one of claims 15 to 17, wherein said sugar plant residue comprises at least one nutrient chosen from

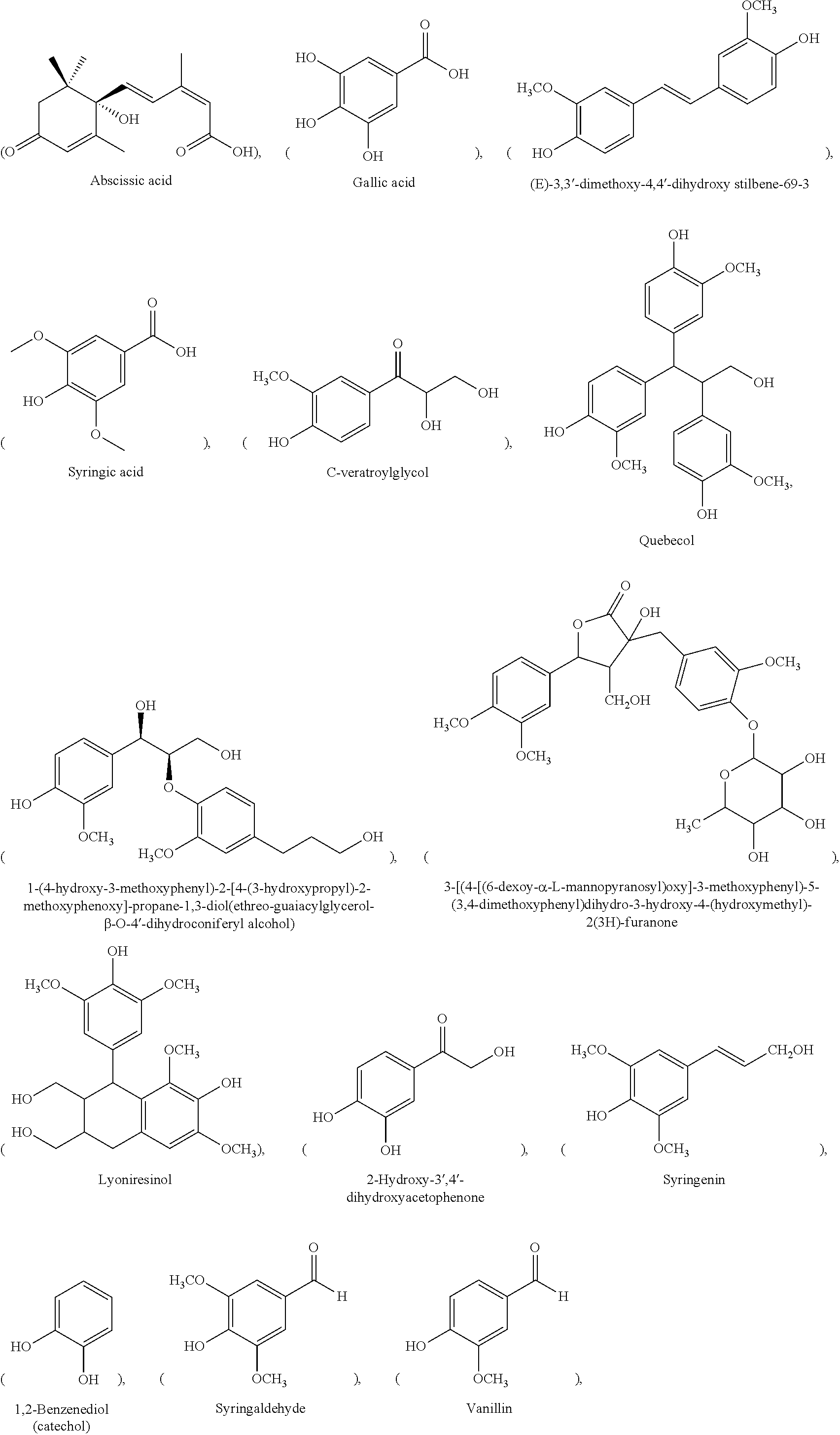

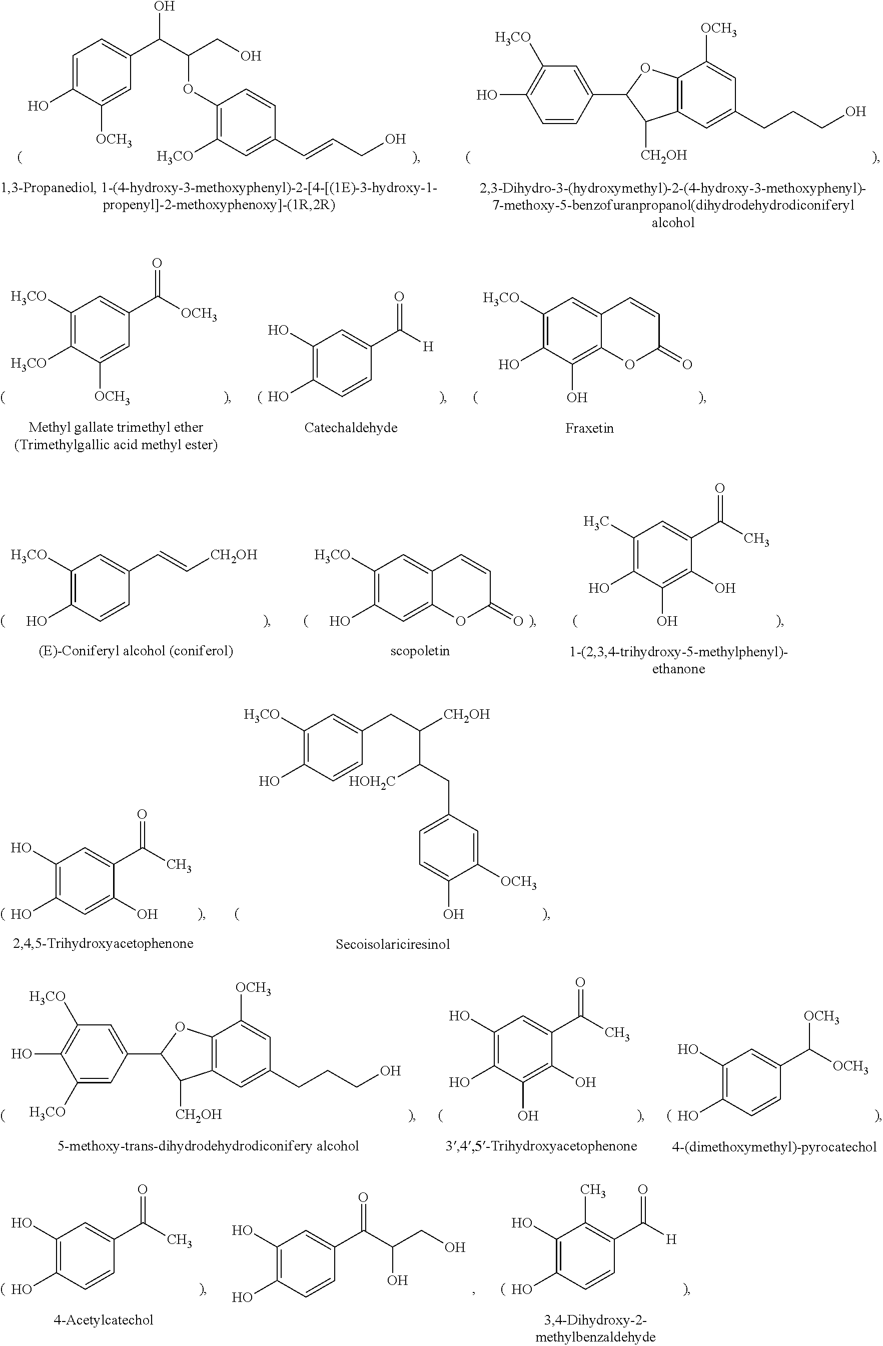

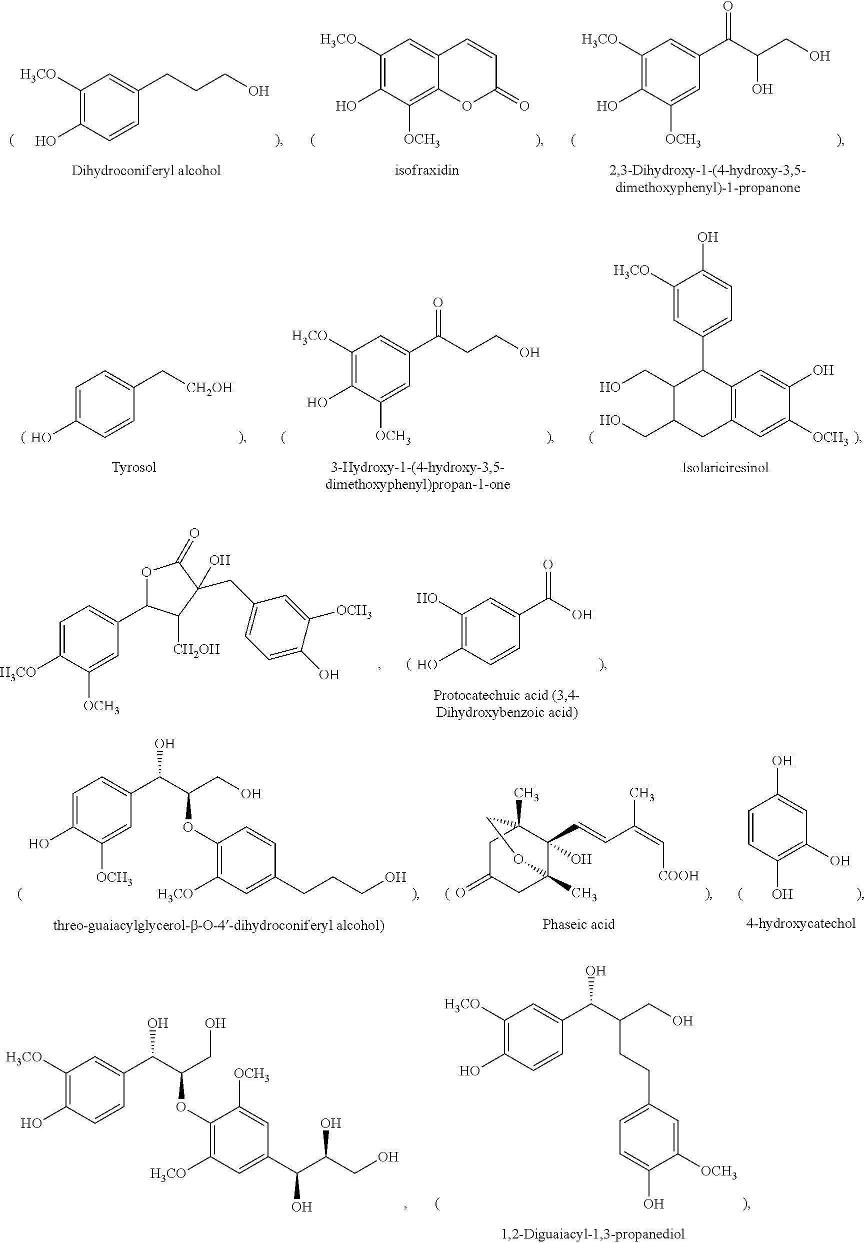

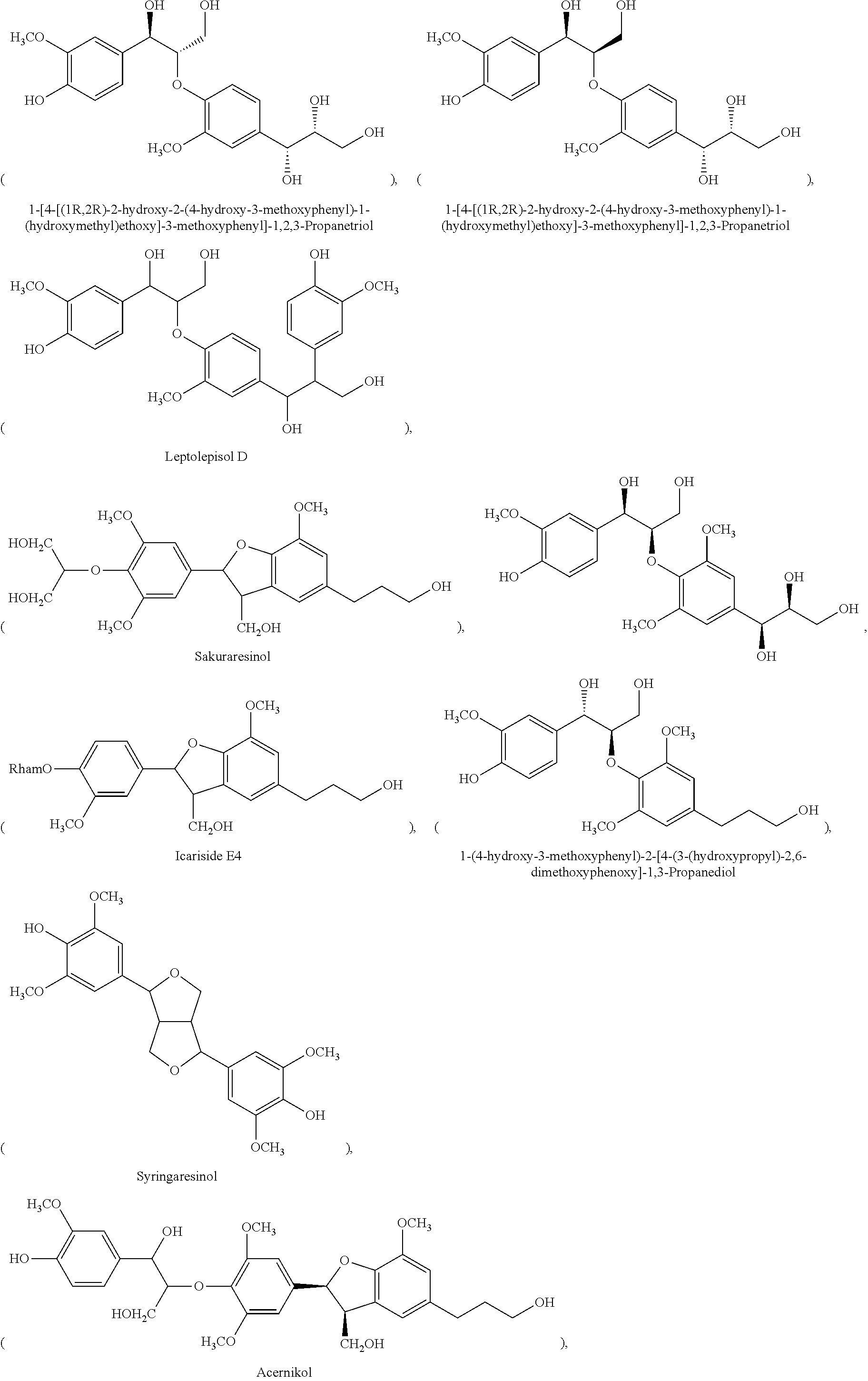

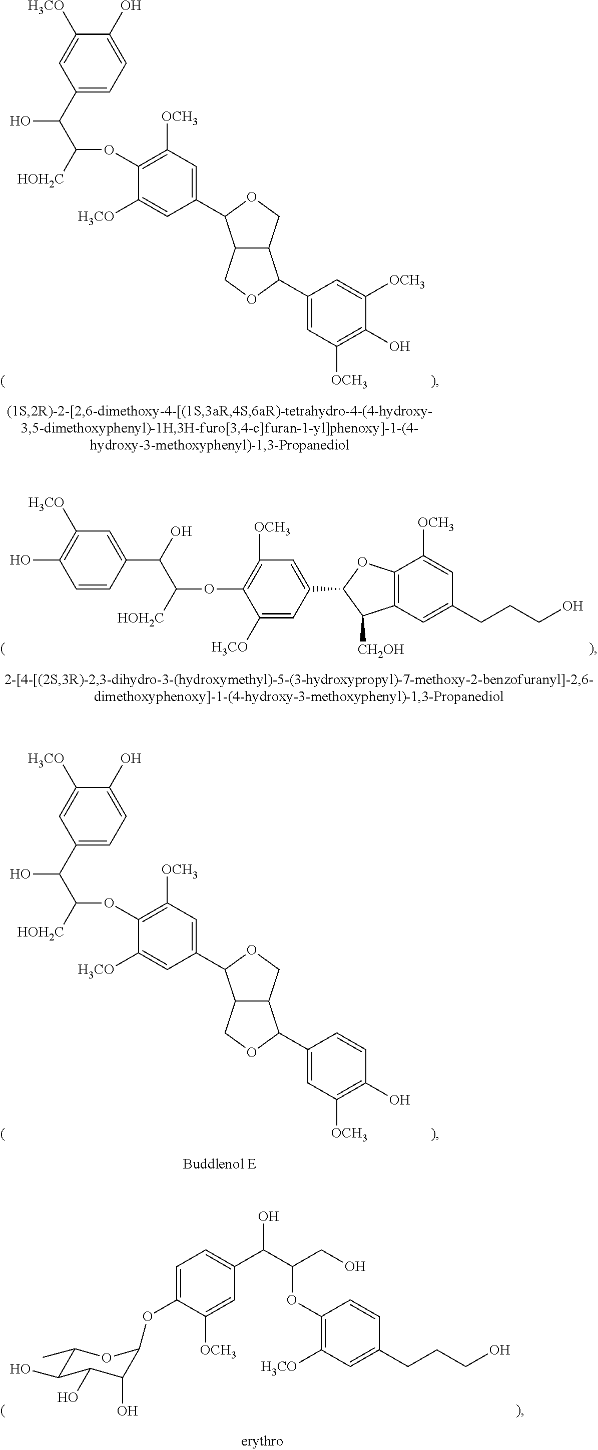 [SMILES: COc1cc(C(O)C(CO)Oc2c(OC)cc(C3OCC4C(c5cc(OC)c(O)c(OC)c5)OCC34)cc2OC)ccc1O ; COc1cc(C(O)C(CO)Oc2c(OC)cc([C@@H]3Oc4c(OC)cc(CCCO)cc4[C@H]3CO)cc2OC)ccc1O ; COc1cc(C(O)C(CO)Oc2c(OC)cc(C3OCC4C(c5ccc(O)c(OC)c5)OCC34)cc2OC)ccc1O ; COc1cc(CCCO)ccc1OC(CO)C(O)c1ccc(OC2OC(C)C(O)C(O)C2O)c(OC)c1]

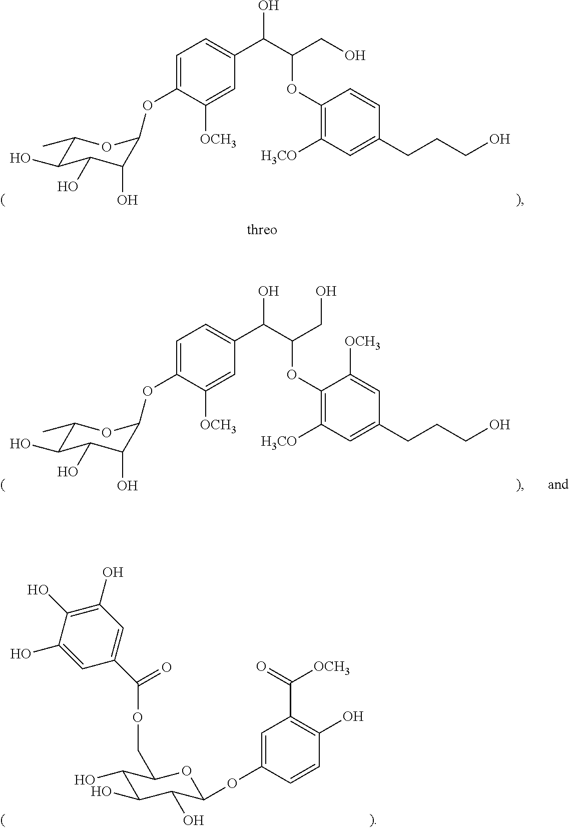 [SMILES: COc1cc(CCCO)ccc1OC(CO)C(O)c1ccc(OC2OC(C)C(O)C(O)C2O)c(OC)c1 ; COc1cc(C(O)C(CO)Oc2c(OC)cc(CCCO)cc2OC)ccc1OC1OC(C)C(O)C(O)C1O ; COC(=O)c1cc(OC2OC(COC(=O)c3cc(O)c(O)c(O)c3)C(O)C(O)C2O)ccc1O]

19. A nutraceutical or cosmeceutical composition according to any one of claims 15 to 18 for the prophylaxis of an ailment comprising a therapeutically effective amount of a sugar plant by-product residue in association with a pharmaceutically acceptable carrier.

20. The nutraceutical or cosmeceutical composition of claim 19, wherein said sugar plant by-product residue is free of a filtration residue.

21. The nutraceutical or cosmeceutical composition of claim 19, wherein said sugar plant by-product residue contains a filtration residue.

22. The nutraceutical or cosmeceutical composition of claim 21, wherein said filtration residue is chosen from diatomaceous earth, celite, kieselguhr, silica, silicon dioxide, calcium, natural sugar sand, ground bones, slop, clay and the like.

23. A topical nutraceutical or cosmeceutical composition according to any one of claims 15 to 18 for improving health condition of skin or various organs comprising a therapeutically effective amount of a sugar plant by-product residue in association with a pharmaceutically acceptable carrier.

24. The topical nutraceutical or cosmeceutical composition of claim 22, wherein said sugar plant by-product residue is free of a filtration residue.

25. The topical nutraceutical or cosmeceutical topical composition of claim 22, wherein said sugar plant by-product residue contains a filtration residue.

26. The topical nutraceutical or cosmeceutical composition of claim 25, wherein said filtration residue is chosen from diatomaceous earth, celite, kieselguhr, silica, silicon dioxide, calcium, natural sugar sand, ground bones, slop, clay and the like.

27. A method of prophylaxis of an ailment comprising administering to a subject in need thereof a nutraceutical or cosmeceutical composition according to any one of claims 19 to 22.

28. The method of claim 27, wherein said ailment is chosen from a metabolic syndrome, diabetes, a cancer, an arthritis, a micro-organism infection, neurodegenerative disease, an inflammation, an inflammatory condition, an oxidative stress related disease, an intestinal dysfunction and a heart disease.

29. The method according to claim 28, wherein said intestinal dysfunction is chosen from an inflammatory bowel disease, Crohn's disease, an ulcerative colitis, collagenous colitis, lymphocytic colitis, ischaemic colitis, diversion colitis, Behçet's disease, and indeterminate colitis.

30. The method according to claim 28, wherein said micro-organism infection is chosen from a bacterial infection, a fungal infection, and combinations thereof.

31. Use of a nutraceutical or cosmeceutical composition according to any one of claims 19 to 22 for the prophylaxis of an ailment.

32. The use of claim 31, wherein said ailment is chosen from a metabolic syndrome, diabetes, a cancer, an arthritis, a micro-organism infection, neurodegenerative disease, an inflammation, an inflammatory condition, an oxidative stress related disease, an intestinal dysfunction and a heart disease.

33. The use according to claim 32, wherein said intestinal dysfunction is chosen from an inflammatory bowel disease, Crohn's disease, an ulcerative colitis, collagenous colitis, lymphocytic colitis, ischaemic colitis, diversion colitis, Behcet's disease, and indeterminate colitis.

34. The use according to claim 32, wherein said micro-organism infection is chosen from a bacterial infection, a fungal infection, and combinations thereof.

35. A food sugar replacement with healthy nutritional values, comprising a nutraceutical or cosmeceutical composition according to any one of claims 15 to 18.

36. The food sugar replacement as claimed in claim 35, wherein said food sugar replacement is a functional food, a food ingredient, an additive, a cosmeto-food, a pharmaceutical, and a food supplement.

37. A non-food ingredient comprising a nutraceutical or cosmeceutical composition according to any one of claims 15 to 18.
